(19) United States
(12) Patent Application Publication
(10) Pub. No.: US 2002/0062079 A1
(43) Pub. Date: May 23, 2002

(54) Composite type ultrasonic probe and a method of manufacturing the same

(75) Inventor: Yoshihiro Tahara, Saitama (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: NIHON DEMPA KOGYO CO., LTD., Tokyo (JP)

(21) Appl. No.: 09/987,858

(22) Filed: Nov. 16, 2001

(30) Foreign Application Priority Data
Nov. 22, 2000 (JP) 2000-355204

(51) Int. Cl. A61B 8/14

(52) U.S. Cl. 600/459

(57) ABSTRACT

A composite type ultrasonic probe in which a plurality of probe bodies are unified together, includes an assembly of a plurality of probe bodies which are unified together to form the assembly, and a casing for housing therein the assembly. Each probe body has a base, backing material, and piezoelectric elements arranged in such a manner that the backing material and the piezoelectric elements are layered on the base, The casing preferably consists of a casing of a single construction and more preferably consists of one formed by integral molding of resin.

### BACKGROUND OF THE INVENTION

1. Field of the Invention

The present invention relates to a ultrasonic transducer probe for medical use, and more particularly to a composite type ultrasonic probe having a piece of probe unit formed by assembling a plurality of probes together, and a method of manufacturing the composite type ultrasonic probe.

2. Description of the Prior Art

For example, in the ultrasonic wave diagnostic apparatus, an ultrasonic probe is used as a unit for transmitting and receiving an ultrasonic wave for obtaining information from a disease portion of a living body. The ultrasonic probes can be classified into several different types depending on their functions and shapes. For example, as one of them, which is suited for detecting ultrasonic echo from a predetermined range of angle when radiating an ultrasonic wave in a fan like manner, there is an ultrasonic wave probe in which a plurality of piezoelectric elements are arranged along a curved line having predetermined curvature while being directed toward outside, and such probe is usually called a convex type probe. To the contrary, an ultrasonic probe having a plurality of piezoelectric elements arranged along a straight line while being directed toward the same direction is called a linear type probe, which is used for radiating ultrasonic wave in only a specified direction.

Further, in a certain type of ultrasonic probe, i.e., in a composite type ultrasonic probe, a plurality of the above-mentioned type of probe elements are unified together to form a piece of probe unit and are -used properly depending on the functions of the respective elements. For example, there is provided a composite type ultrasonic probe, which consists of a convex type probe and a linear type probe and in which the convex type probe and the linear type probe are unified together to form a piece of probe unit. Such ultrasonic probe is used to be inserted in a body cavity of an examined person or a patient.

FIG. 1 illustrates a main portion of a convex type probe according to the prior art, and FIG. 2 illustrates a main portion of a linear type probe according to the prior art.

Body 1 of the convex type probe uses base 5 having a surface formed in an arc-shape curved face, and is provided with a plurality of piezoelectric elements 2 arranged on the arc-shape surface via backing material 4. On the other hand, body 3 of the linear type probe uses base 15 in the shape of an elongated flat plate, and is provided with a plurality of piezoelectric elements 2 arranged on one of the major surfaces of base 15 via backing material 4. In any one of the probes, piezoelectric element 2 has excitation electrodes (not shown) on its opposite major surfaces, and is fixedly secured to backing material 4. Backing material 4 is fixed to base 5 or 15 at its own surface.

In probe bodies 1 and 3, the excitation electrodes of piezoelectric elements 2 are externally conducted by means of flexible printed board 6. The surface of each piezoelectric element 2, which forms a face for transmitting and receiving ultrasonic wave, is provided with an acoustic matching layer (not shown) formed thereon.

When these convex type probe body 1 and linear type probe body 3 are constitutionally unified together, a composite type ultrasonic probe can be obtained. In such composite type ultrasonic probe of the prior art, the convex type probe body and the linear type probe body are housed in their individual casings, and these casings are bonded together by adhesive.

FIG. 3 illustrates the above-described composite type ultrasonic probe of the prior art. Thus, for example, convex type probe body 1 is mounted in first casing 7 having a headed end and linear type probe body 3 is mounted in a front end of second arced and elongated casing 8 in a manner such that the wave transmitting and receiving faces of the two probe bodies are arranged on the same side. The rear end of second casing 8 is provided with large arced grasping portion 9 formed therein. After first and second casings 7 and 8 are bonded together by e.g., adhesive, and when third casing 10 forming a cover is connected to the bonded casings, convex type probe body 1 and linear type probe body 3 are sealed in an assembly of casings 7, 8 and 10. Flexible printed boards 6 are connected to a non-illustrated cable and are led outside via grasping portion 9.

When the above-constituted composite type ultrasonic probe is inserted, at its headed end, inside a body cavity, information acquired by the ultrasonic wave radiated fan-like manner from convex type probe body 1 and another information acquired by the ultrasonic wave radiated from linear type probe body 3 are used for obtaining diagnostic information of a living body.

Nevertheless, in the composite type ultrasonic probe of the described constitution, since first casing 7 housing therein convex type probe body 1 and second casing 8 housing therein linear type probe body 3 are assembled together by connection, the strength of the connection might be insufficient. As a result when the probe is inserted without attention in the body cavity of a living body such as a patient to conduct diagnosing of the living body, the connection of the casings might be broken. Further, the accuracy of the relative position of a plurality of probe bodies which are assembled with each other might be deteriorated due to the complicated structure of the composite type ultrasonic probe.

### SUMMARY OF THE INVENTION

Therefore, a first object of the present invention is to provide a composite type ultrasonic probe capable of maintaining a physical strength and being gentle for the living body.

A second object of the present invention is to provide a method of manufacturing a composite type ultrasonic probe capable of maintaining a physical strength and being gentle for the living body.

The first object of the present invention can be achieved by a composite type ultrasonic probe, which comprises an assembly of a plurality of probe bodies which are unified together to form the assembly, and a casing for housing therein the assembly. Each of the probe bodies comprises a base, backing material and piezoelectric elements arranged in such a manner that the backing material and the piezoelectric elements are layered on the base.

The casing should preferably be of a single construction, and is preferably formed by integral molding of resin. In other words, the casing is preferably a monolithic member of resin. Since the resin-molded construction can eliminate any less strong connected portion from the casing, the strength of the composite type ultrasonic probe can be enhanced.

In the present invention, the plurality of probes constituting the assembly are, for example, a convex type probe body and a linear type probe body which are bonded together to form the assembly.

The second object of the present invention is achieved by a method of manufacturing a composite type ultrasonic probe, which comprises the steps of: unifying together a plurality of probe bodies to form an assembly, and fitting said assembly in a casing of a single construction, which has therein an opening portion corresponding to the assembly. Each of the probe bodies comprises a base, backing material, and piezoelectric elements arranged in such a manner that the backing material and the piezoelectric elements are layered on the base.

### BRIEF DESCRIPTION OF THE DRAWINGS

FIG. 1 is a perspective view of a convex type probe body according to the prior art;

FIG. 2 is a perspective view of a linear type probe body according to the prior art;

FIG. 3 is an exploded view of a composite type ultrasonic probe according to the prior art;

FIG. 4 is a perspective view of an assembly in an ultrasonic probe according to a preferred embodiment of the present invention; and

FIG. 5 is an exploded view of the ultrasonic probe according to the preferred embodiment of the present invention.

### DETAILED DESCRIPTION OF THE PREFERRED EMBODIMENT

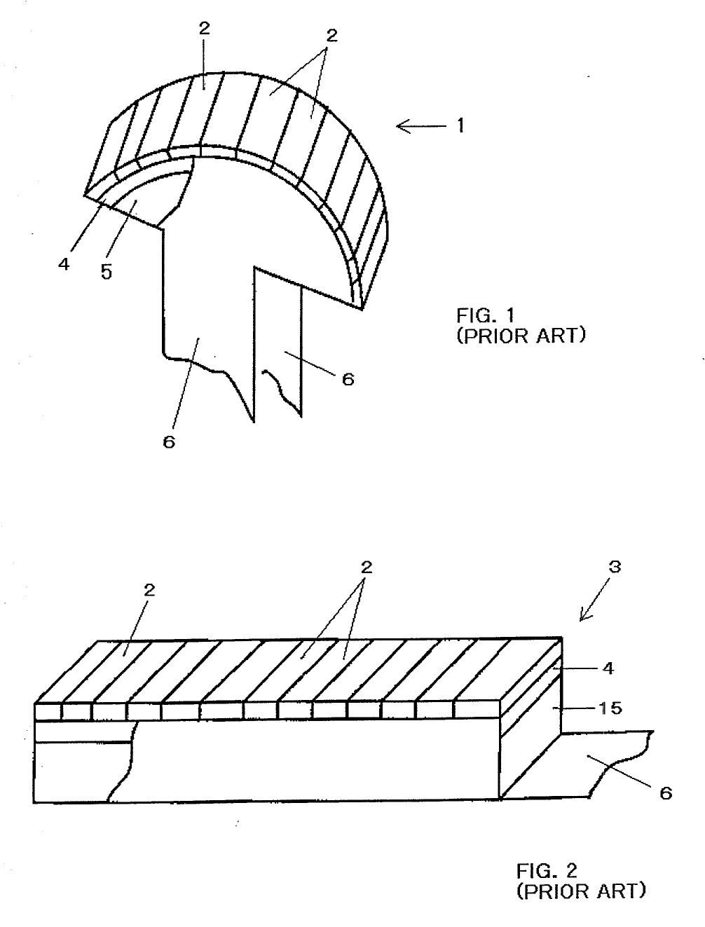

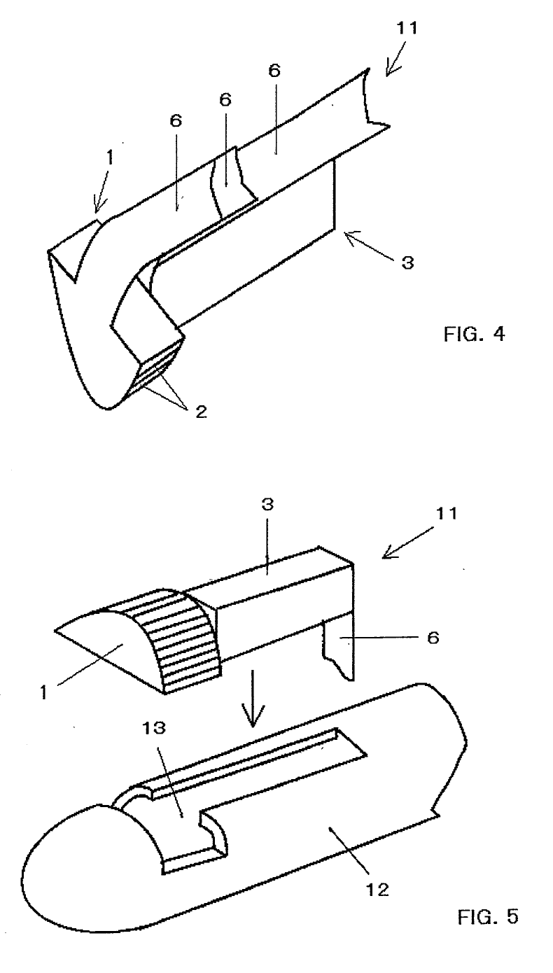

In FIGS. 4 and 5 illustrating a composite type ultrasonic probe according to the preferred embodiment of the present invention, the constituting elements of the probe designated by the same reference numerals as those in FIGS. 1 to 3 are the same as those in FIGS. 1 to 3.

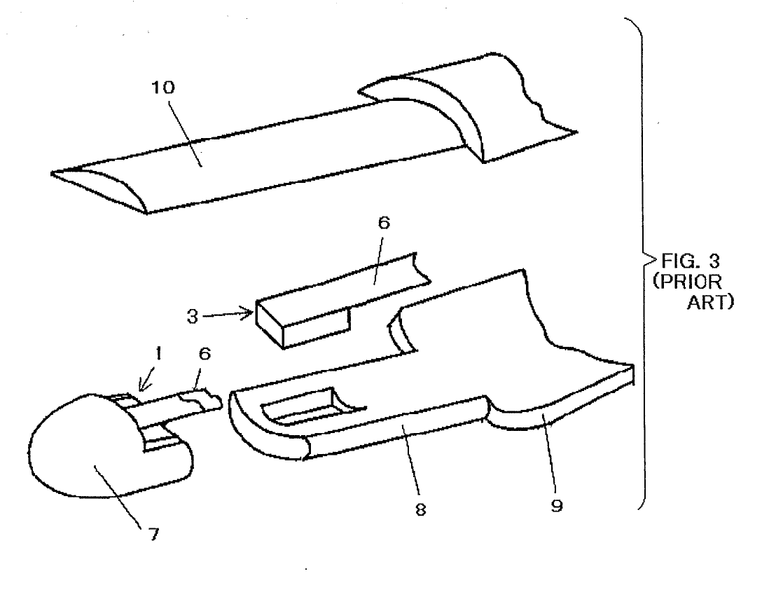

The composite type ultrasonic probe of the preferred embodiment includes convex type probe body 1 having piezoelectric elements 2 arranged on a curved surface, and linear type probe body 3 having piezoelectric elements 2 arranged on a flat face. Convex type probe body 1 and linear type probe body 3 are preliminarily unified together to constitute assembly 11 as shown in FIG. 4. Convex type probe body 1 may consist of one shown in FIG. 1, and linear type probe body 3 may consist of one shown in FIG. 2. The faces of both probe bodies 1 and 3 for transmitting and receiving ultrasonic wave are directed toward the same direction. In the illustrated assembly 11, probe bodies 1 and 3, from which flexible printed boards 6 are led toward the outside, are bonded together by adhesive to be unified together, and flexible printed boards 6 are bundled together on the back side of the bases of respective probe bodies 1 and 3, and are led to the rear side of the same bases. The bonding of probe bodies 1 and 3 is performed by the use of a jig or a mounting piece which is accommodated in a casing with the prove bodies so as to enhance the bonding accuracy of both probe bodies.

Thus constituted assembly 11 is fitted in casing 12. Casing 12 may be acquired, for example, by the integral molding of resin, and is formed in an elongated shape to have a headed front end. Casing 12 is also formed to have opening portion 13 in one of major faces thereof so that the shape of the opening portion corresponds to that of assembly 11. Although not illustrated in FIG. 5, casing 12 is provided, at its rear end, with a large diameter-grasping portion similar to that shown in FIG. 3.

Assembly 11 is fitted in the above-mentioned opening portion 13 of casing 12 so that the faces on which piezoelectric elements 2 of each of probes 1 and 3 are exposed toward the outside, and is bonded to casing 12 by adhesive. Flexible printed boards 6 led to the rear side of casing 12 are electrically connected to a non-illustrated cable.

As described in the foregoing, in the present embodiment, convex type probe body 1 and linear type probe body 3 are preliminarily unified together to form assembly 11, and this assembly 11 is accommodated in casing 12 of a single construction so as to constitute a composite type ultrasonic probe. Therefore, in the completed convex type ultrasonic probe, since casing 12 has therein no connected portion that is less strong, the strength of casing 12 per se can be appreciably enhanced. Thus, when the composite type ultrasonic probe is inserted in a body cavity of a living body such as a patient to diagnose the living body, there is not any anxiety about breakage of casing 12, and accordingly the living body can be prevented from being wounded while enhancing the safety of the probe.

In the foregoing description, although convex type probe body 1 and linear type probe body 3 are unified together to constitute assembly 11, it is to be understood that the present invention is not limited to this embodiment. AS is well known, the ultrasonic probe may be of a single type or a sector type other than the convex type and the linear type. Thus, the present invention may be applicable to a case where these many types of probe bodies are combined together to constitute a composite type ultrasonic probe. For example, a sector type probe body and the linear type probe body may be first unified together to form an assembly, and subsequently the assembly may be fitted in a casing of a single construction to form a composite type ultrasonic probe. Further, the present invention may be applicable to a case where a plurality of probe bodies of the same type are combined together to constitute a composite type ultrasonic probe.

What is claimed is:

1. A composite type ultrasonic probe comprising:
an assembly of a plurality of probe bodies which are unified together to form the assembly, each of said probe bodies comprising a base, backing material and piezoelectric elements arranged in such a manner that the backing material and the piezoelectric elements are layered on said base; and
a casing for housing therein said assembly.

2. A composite type ultrasonic probe according to claim 1, wherein said casing comprises a casing of a single construction.

3. A composite type ultrasonic probe according to claim 2, wherein said casing is comprised of one formed by integral molding of resin.

4. A composite type ultrasonic probe according to claim 3, wherein said casing has a surface thereof formed therein an opening portion for accommodating therein said assembly.

5. A composite type ultrasonic probe according to claim 1, wherein said assembly comprises a convex type probe body and a linear type probe body bonded together.

6. A method of manufacturing a composite type ultrasonic probe comprising the steps of:
unifying together a plurality of probe bodies to form an assembly, each of said probe bodies comprising a base, backing material, and piezoelectric elements arranged in such a manner that the backing material and the piezoelectric elements are layered on said base; and
fitting said assembly in a casing of a single construction, which has therein an opening portion corresponding to said assembly.

7. A method according to claim 6, further comprising a step of forming said casing by integral molding of resin.

8. A method according to claim 6, wherein said assembly is constituted by boding a convex type probe body and a linear type probe body together.
